(19) United States
(12) Patent Application Publication
(10) Pub. No.: US 2026/0047884 A1
(43) Pub. Date: Feb. 19, 2026

(54) CLOSED-LOOP ABLATION THERAPY

(71) Applicant: St. Jude Medical, Cardiology Division, Inc., St. Paul, MN (US)

(72) Inventors: Travis Dahlen, Forest Lake, MN (US); Sina Farahmand, Woodbury, MN (US)

(21) Appl. No.: 19/278,153

(22) Filed: Jul. 23, 2025

Related U.S. Application Data
(60) Provisional application No. 63/683,327, filed on Aug. 15, 2024.

(51) Int. Cl. A61B 18/14; A61B 18/00

(52) U.S. Cl. CPC A61B 18/1492; A61B 2018/00351; A61B 2018/00577; A61B 2018/00648; A61B 2018/00702; A61B 2018/00708; A61B 2018/00773; A61B 2018/00839; A61B 2018/00875

(57) ABSTRACT

An ablation system includes an ablation generator, catheter, and a closed-loop ablation processor operably coupled thereto. The processor receives baseline tissue properties, such as baseline tissue impedance, tissue thickness, tissue anatomical location, tissue type, catheter-tissue contact force, catheter stability against the tissue, and baseline electrogram signal morphology. The processor also receives waveform parameters for the generator. Using the baseline tissue properties and the waveform parameters, the processor determines whether pulsed field or radiofrequency ablation therapy should be delivered and computes corresponding target ablation metric(s), such as a target ablation index. The processor then commands the generator to deliver ablation therapy to the tissue and monitors lesion formation using intra-therapeutic tissue properties (and waveform parameters) to compute intra-therapeutic ablation metric(s) and comparing the intra-therapeutic ablation metric(s) to the target ablation metric(s). Ablation therapy ceases when the intra-therapeutic ablation metric(s) satisfy the target ablation metric(s).

### CROSS-REFERENCE TO RELATED APPLICATIONS

The present application claims the benefit of U.S. provisional application No. 63/683,327, filed 15 Aug. 2024, which is hereby incorporated by reference as though fully set forth herein.

### FIELD

The present disclosure relates generally to electrophysiology procedures. In particular, the present disclosure relates to methods and systems for the delivery of closed-loop ablation therapy, including dual-mode ablation therapy, to a tissue.

### BACKGROUND

Ablation therapy may be used to treat various conditions afflicting the human anatomy. One such condition in which ablation therapy may be used is the treatment of cardiac arrhythmias. When tissue is ablated, or at least subjected to ablative energy generated by an ablation generator and delivered by an ablation catheter, lesions form in the tissue. Electrodes mounted on or in ablation catheters are used to create tissue necrosis in cardiac tissue to correct conditions such as atrial arrhythmia (including, but not limited to, ectopic atrial tachycardia, atrial fibrillation, and atrial flutter). Arrhythmias can create a variety of dangerous conditions including loss of synchronous atrioventricular contractions and stasis of blood flow. It is believed that the primary cause of atrial arrhythmias is stray electrical signals within the left or right atrium of the heart. The ablation catheter imparts ablative energy (e.g., radiofrequency energy, cryoablation, lasers, chemicals, high-intensity focused ultrasound, etc.) to cardiac tissue to create a lesion in the cardiac tissue. This lesion disrupts undesirable electrical pathways and thereby limits or prevents stray electrical signals that may lead to arrhythmias.

One form of ablation therapy is radiofrequency ablation (RFA). In RFA, heat produced by radiofrequency (RF) energy is used to destroy tissue and create transmural lesions.

Electroporation is a non-thermal ablation technique that involves applying strong electric fields that induce pore formation in the cellular membrane. The electric field may be induced by applying a relatively short duration pulse which may last, for example, from a nanosecond to several milliseconds. Such a pulse may be repeated to form a pulse train (or “burst”). When such an electric field is applied to tissue in an in vivo setting, the cells in the tissue are subjected to a trans-membrane potential, which opens the pores on the cell wall. Electroporation may be reversible (i.e., the temporarily-opened pores will reseal) or irreversible (i.e., the pores will remain open, causing cellular destruction). In certain therapeutic applications, a suitably configured pulse train alone may be used to cause cell destruction, for instance by causing irreversible electroporation (IRE). In the context of cardiac ablation to create transmural lesions, this is known as pulsed field ablation (PFA).

In certain contexts, PFA may be more advantageous than RFA. For instance, the non-thermal effects of PFA may be desirable in certain anatomical locations (e.g., in most left atrial locations), and PFA may also be faster than RFA in creating transmural lesions to interrupt errant conduction pathways. On the other hand, in other anatomical locations, such as atrial locations adjacent to coronary arteries, RFA may be more suitable than PFA.

Moreover, extant PFA systems, and most extant RFA systems, deliver predetermined (or “open-loop”) therapy. For instance, in an open-loop PFA system, the number of PFA bursts may be fixed, rather than specifically tailored to real-world circumstances (e.g., patient anatomy, tissue response, and so on).

### BRIEF SUMMARY

The instant disclosure provides a method of delivering closed-loop ablation therapy to a tissue via an ablation system including an ablation generator and a closed-loop ablation processor operably coupled to the ablation generator. The method includes: receiving baseline properties of the tissue as inputs to the closed-loop ablation processor, the baseline properties of the tissue including one or more of a baseline tissue impedance, a tissue thickness, a tissue anatomical location, a tissue type, a contact force between the ablation catheter and the tissue, a stability of the ablation catheter against the tissue, and a baseline electrogram (EGM) signal morphology; receiving ablation waveform parameters for the ablation generator as inputs to the closed-loop ablation processor; the closed-loop ablation processor computing a target ablation metric using the baseline properties of the tissue and the ablation waveform parameters; after computing the target ablation metric, the closed-loop ablation processor commanding the ablation generator to deliver ablation therapy to the tissue; after commanding the ablation generator to deliver ablation therapy to the tissue, the closed-loop ablation processor executing a lesion formation monitoring process including receiving intra-therapeutic properties of the tissue as input, the intra-therapeutic properties of the tissue including one or more of an intra-therapeutic tissue impedance, an intra-therapeutic contact force, an intra-therapeutic catheter stability, and an intra-therapeutic EGM signal morphology; computing an intra-therapeutic ablation metric using the intra-therapeutic properties of the tissue; and comparing the intra-therapeutic ablation metric to the target ablation metric; and the closed-loop ablation processor commanding the ablation generator to cease delivering ablation therapy to the tissue when the intra-therapeutic ablation metric satisfies the target ablation metric.

The target ablation metric can include one or more of a target ablation index, a target impedance drop, a target radiofrequency ablation (RFA) duration, and a target EGM signal morphology variation. The target ablation index may be computed according to a multivariate regression model, such as a multivariate logistic regression model, wherein variables of the multivariate regression model correspond to one or more of the baseline properties of the tissue and the ablation waveform parameters.

In further embodiments of the disclosure, the method also includes, prior to the closed-loop ablation processor commanding the ablation generator to deliver ablation therapy to the tissue, the closed-loop ablation processor determining, based on the baseline properties of the tissue, either to command the ablation generator to deliver RFA therapy to the tissue or to command the ablation generator to deliver pulsed field ablation therapy to the tissue. For instance, the closed-loop ablation processor can determine either to command the ablation generator to deliver RFA therapy to the tissue or to command the ablation generator to deliver pulsed field ablation therapy to the tissue based on one or more of the tissue thickness, the tissue anatomical location, and the tissue type.

The intra-therapeutic properties of the tissue can further include one or more of tissue thickness, tissue anatomical location, and tissue type.

According to aspects of the disclosure, the ablation therapy includes pulsed field ablation therapy and the closed-loop ablation processor executes the lesion formation monitoring process during intervals between successive bursts of pulsed field ablation therapy. The pulsed field ablation therapy may also be R-wave synchronized. Further, the closed-loop ablation processor can command the ablation generator to suspend delivery of ablation therapy to the tissue for a preset safety interval after a preset number of successive bursts of pulsed field ablation therapy are delivered to the tissue.

Also disclosed herein is a closed-loop ablation system including an ablation generator; an ablation catheter operably coupled to the ablation generator; and a closed-loop ablation processor operably coupled to the ablation generator. The closed-loop ablation processor is configured to: receive baseline properties of a tissue to be ablated as inputs, the baseline properties of the tissue including one or more of: a baseline tissue impedance, a tissue thickness, a tissue anatomical location, a tissue type, a contact force between the ablation catheter and the tissue, a stability of the ablation catheter against the tissue, and a baseline electrogram (EGM) signal morphology; receive ablation waveform parameters for the ablation generator as inputs; compute a target ablation metric using the baseline properties of the tissue and the ablation waveform parameters; command the ablation generator to deliver ablation therapy to the tissue after computing the target ablation metric; execute a lesion formation monitoring process after commanding the ablation generator to deliver ablation therapy to the tissue, the lesion formation monitoring processing including receiving intra-therapeutic properties of the tissue as inputs, the intra-therapeutic properties of the tissue including one or more of an intra-therapeutic tissue impedance, an intra-therapeutic contact force, an intra-therapeutic catheter stability, and an intra-therapeutic EGM signal morphology; computing an intra-therapeutic ablation metric using the intra-therapeutic properties of the tissue; and comparing the intra-therapeutic ablation metric to the target ablation metric; and command the ablation generator to cease delivering ablation therapy to the tissue when the intra-therapeutic ablation matric satisfies the target ablation metric.

The target ablation metric can include one or more of a target ablation index, a target impedance drop, a target radiofrequency ablation (RFA) duration, and a target EGM signal morphology variation. The target ablation index may be computed according to a multivariate regression model, such as a multivariate logistic regression model, wherein variables of the multivariate regression model correspond to one or more of the baseline properties of the tissue and the ablation waveform parameters.

The closed-loop ablation processor may also be configured to determine, based on the baseline properties of the tissue, either to command the ablation generator to deliver RFA therapy to the tissue or to command the ablation generator to deliver pulsed field ablation therapy to the tissue. For instance, the closed-loop ablation processor may be configured to determine, based on at least one of the tissue thickness, the tissue anatomical location, and the tissue type, either to command the ablation generator to deliver RFA therapy to the tissue or to command the ablation generator to deliver pulsed field ablation therapy to the tissue.

According to aspects of the disclosure, the ablation therapy includes pulsed field ablation therapy and the closed-loop ablation processor executes the lesion formation monitoring process during intervals between successive bursts of pulsed field ablation therapy. The pulsed-field ablation therapy may also be R-wave synchronized. In still further aspects of the disclosure, the closed-loop ablation processor may be configured to command the ablation generator to suspend delivery of the ablation therapy to the tissue for a preset safety interval after a preset number of successive bursts of pulsed field ablation therapy are delivered to the tissue.

The intra-therapeutic properties of the tissue can further include the tissue thickness, the tissue anatomical location, and the tissue type.

The instant disclosure also provides a method of delivering closed-loop ablation therapy to a tissue via a dual-mode ablation system including an ablation generator, an ablation catheter operably coupled to the ablation generator, and a closed-loop ablation processor operably coupled to the ablation generator. The method includes: the closed-loop ablation processor receiving baseline properties of the tissue, the baseline properties of the tissue including one or more of: a baseline tissue impedance, a tissue thickness, a tissue anatomical location, a tissue type, a contact force between the ablation catheter and the tissue, a stability of the ablation catheter against the tissue, and a baseline electrogram (EGM) signal morphology; the closed-loop ablation processor receiving ablation waveform parameters for the ablation generator; the closed-loop ablation processor selecting between radiofrequency ablation therapy and pulsed field ablation therapy based on the baseline properties of the tissue; the closed-loop ablation processor computing a target ablation index using a multivariate regression model wherein variables of the multivariate regression model correspond to one or more of the baseline properties of the tissue and one or more of the ablation waveform parameters; and the closed-loop ablation processor titrating delivery of the selected ablation therapy by monitoring a relationship between an intra-therapeutic ablation index and the target ablation index, wherein the intra-therapeutic ablation index is a function of one or more intra-therapeutic properties of the tissue.

Optionally, the method may also include the closed-loop ablation processor titrating delivery of the selected ablation therapy by monitoring a drop in tissue impedance relative to the baseline tissue impedance.

In still further embodiments, the disclosure provides a method of delivering closed-loop ablation therapy to a tissue via an ablation system including an ablation generator and a closed-loop ablation processor operably coupled to the ablation generator. The method includes: receiving baseline properties of the tissue as inputs to the closed-loop ablation processor, the baseline properties of the tissue including one or more of a baseline tissue impedance, a tissue thickness, a tissue anatomical location, a tissue type, a contact force between the ablation catheter and the tissue, a stability of the ablation catheter against the tissue, and a baseline electrogram (EGM) signal morphology; and the closed-loop ablation processor determining, based on the baseline properties of the tissue, either to command the ablation generator to deliver radiofrequency ablation therapy to the tissue or to command the ablation generator to deliver pulsed field ablation therapy to the tissue, and then commanding the ablation generator to deliver the determined ablation therapy to the tissue.

There is also provided a computer readable medium, a record carrier or a computer program product comprising instructions that, when executed, cause a computer or processor to perform any of the methods set forth herein.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1A is a diagrammatic and block diagram view of an illustrative system for closed-loop ablation therapy, including dual-mode ablation therapy.

FIG. 1B is a schematic representation of an ablation catheter for use in connection with the closed-loop ablation therapy system of FIG. 1A.

FIG. 2A is a flowchart of representative steps that can be carried out according to aspects of the instant disclosure.

FIG. 2B illustrates inputs to and outputs from a closed-loop ablation processor according to aspects of the present disclosure.

FIG. 3 is a flowchart illustrating a lesion formation monitoring process that can be followed in embodiments of the disclosure where PFA therapy is being delivered to a tissue. Optional steps are shown in dashed lines.

FIG. 4 is a flowchart illustrating a lesion formation monitoring process that can be followed in embodiments of the disclosure where RFA therapy is being delivered to a tissue.

While multiple embodiments are disclosed, still other embodiments of the present disclosure will become apparent to those skilled in the art from the following detailed description, which shows and describes illustrative embodiments. Accordingly, the drawings and detailed description are to be regarded as illustrative in nature and not restrictive.

### DETAILED DESCRIPTION

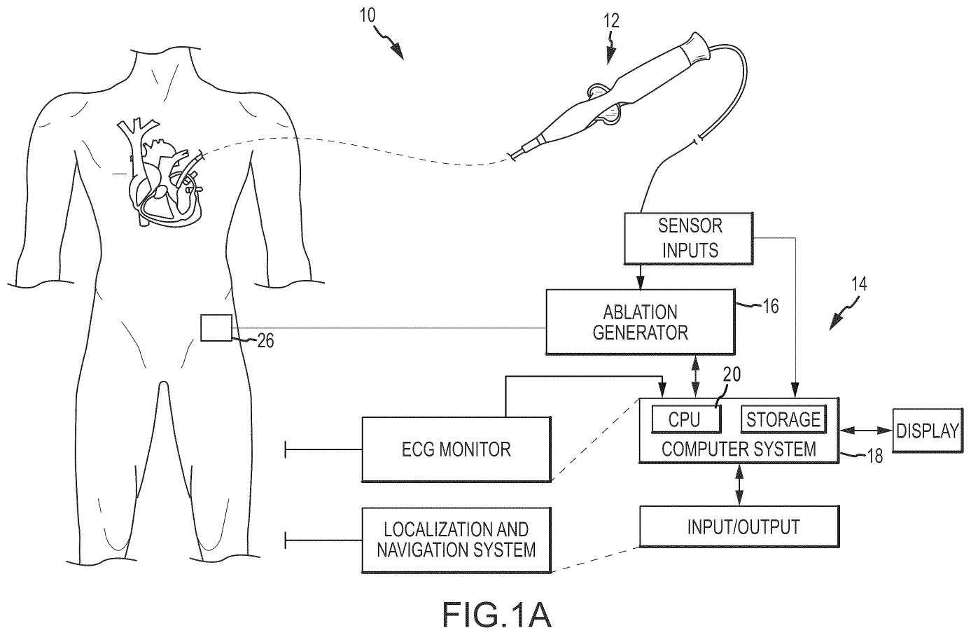

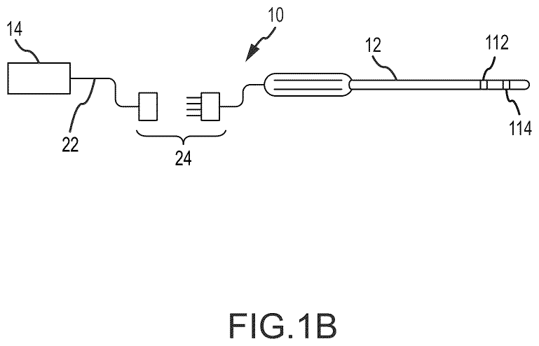

FIG. 1A is a diagrammatic and block diagram view of a closed-loop ablation system 10. Closed-loop ablation system 10 may be used for RFA and/or PFA to destroy tissue and create lesions for the treatment of various arrhythmias.

For purposes of illustration, aspects of the disclosure will be described with reference to a dual-mode, closed-loop ablation system. The term “dual-mode” is used herein to refer to an ablation system that is capable of delivering both RFA therapy and PFA therapy to a tissue (though not necessarily simultaneously or in the same procedure) (which is analogously referred to herein as “dual-mode ablation therapy”). Conversely, a closed-loop ablation system only capable of delivering either RFA therapy or PFA therapy to a tissue, but not both, would be referred to as a “single-mode ablation system” (and would deliver “single-mode ablation therapy”). Those of ordinary skill in the art will, however, appreciate that the teachings herein may be applied to good advantage not only in the context of dual-mode ablation systems, but also in the context of single-mode ablation systems. Moreover, insofar as those of ordinary skill in the art will be generally familiar with ablation systems and RFA and PFA therapies, they will not be discussed in detail herein except to the extent necessary to understand the instant disclosure.

In general, closed-loop ablation system 10 includes an ablation catheter 12, such as the TactiFlex™ Duo Ablation Catheter, Sensor Enabled™ (Abbott Laboratories; Abbott Park, Illinois), and various electronics 14 (e.g., ablation generator 16; computer system 18 including closed-loop ablation processor 20 and storage and connected to a display and input/output devices; an electroanatomical mapping system; and so on). As illustrated in FIG. 1A, both ablation catheter 12 and closed-loop ablation processor 20 are operably coupled to ablation generator 16.

FIG. 1B schematically illustrates ablation catheter 12. Ablation catheter 12 includes one or more electrodes 112, 114 (collectively referred to herein as an “electrode assembly”), which may be used for a variety of diagnostic and/or therapeutic purposes including, without limitation, electrophysiological mapping, RFA therapy, and/or PFA therapy. For example, and in some embodiments of the disclosure, the electrode assembly may be configured as a bipolar electrode assembly for use in bipolar-based PFA therapy. Specifically, electrodes 112, 114 may be individually electrically coupled to ablation generator 16 (e.g., via suitable electrical wire 22 or other suitable electrical conductors connected through electrical connector 24 as discussed in further detail below) and configured to be selectively energized (e.g., by ablation generator 16 under control of closed-loop ablation processor 20) with opposite polarities to generate a potential and corresponding electric field therebetween for PFA therapy. That is, one of electrodes 112, 114 can be configured to function as a cathode, and the other can be configured to function as an anode for a given PFA burst.

Electrodes 112, 114 may be any suitable electrodes. In an exemplary embodiment, electrodes 112, 114 are ring electrodes, though electrodes 112, 114 may have any other shape or configuration without departing from the scope of the present disclosure. Moreover, although each of electrode 112 and electrode 114 are illustrated as single electrodes, either or both of electrode 112 and electrode 114 may be alternatively embodied as two or more discrete electrodes.

Those of ordinary skill in the art will recognize that variations in the shape, size, and/or configuration of electrodes 112, 114 may result in variations in parameters of the applied ablation therapy. For example, increasing the surface area of one or both electrodes 112, 114 would decrease impedance, in turn decreasing the current that would need to be applied to achieve the voltage level required to cause tissue destruction in PFA therapy.

Further, while the electrode assembly is described as a bipolar electrode assembly, it should be understood that, in some embodiments, the electrode assembly may be configured as a unipolar electrode assembly and use a patch electrode on the patient's skin (e.g., 26) as a return or indifferent electrode for use in monopolar-based PFA therapy.

Also shown in FIG. 1B is an electrical connector 24. As shown, the plug side of electrical connector 24 is connected to catheter 12, while the receptacle side of electrical connector 24 is connected to electronics 14 via cable 22. Of course, this arrangement could be reversed without departing from the scope of the instant disclosure. The ordinarily skilled artisan will appreciate that, when the plug and receptacle portions of electrical connector 24 are mated, catheter 12 becomes electrically coupled to electronics 14, enabling power, data, and other electrical signals to pass between the two.

Computer system 18 may comprise, for example, a conventional general-purpose computer, a special-purpose computer, a distributed computer, or any other type of computer. Computer system 18 may include one or more processors, such as a single central processing unit (“CPU”), or a plurality of processing units, commonly referred to as a parallel processing environment, which may execute instructions to practice the various aspects described herein. In particular, aspects of the disclosure may be executed via a closed-loop ablation processor 20, as further described below. As will be appreciated, ablation processor 20 is configured to process data, and so the inputs to ablation processor 20 may be digitized into data representative of the input, such that ablation processor 20 may process that data.

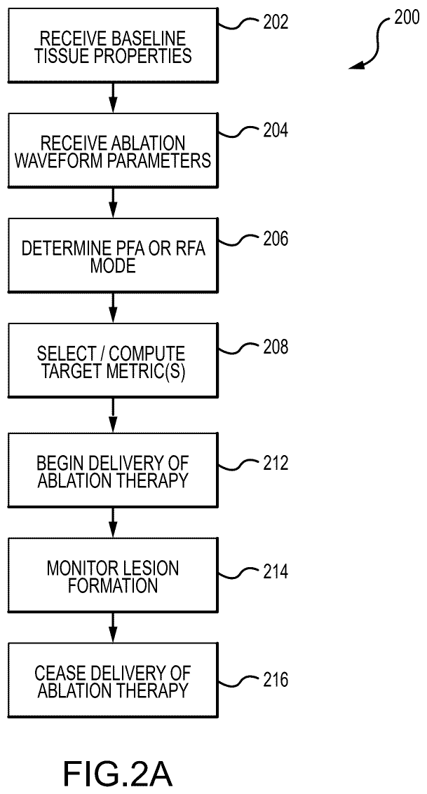

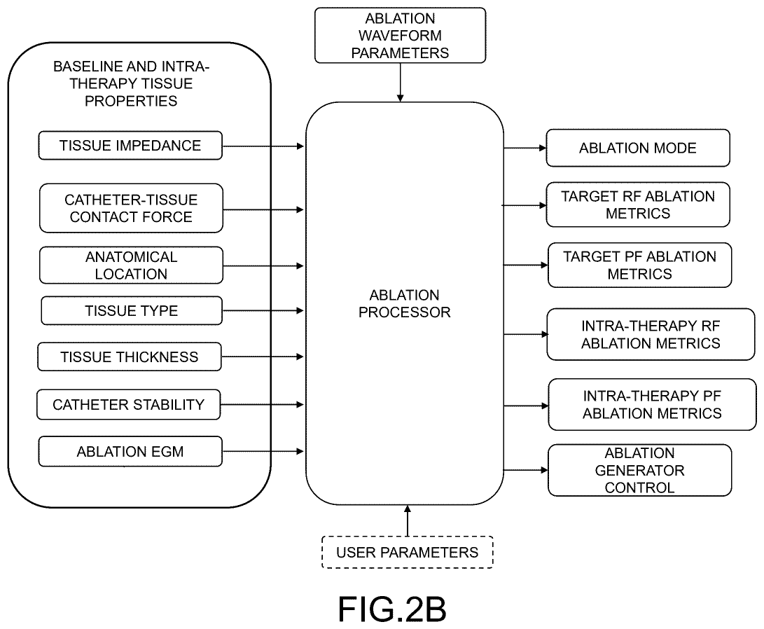

Exemplary methods according to aspects of the instant disclosure will be explained with reference to the flowchart 200 of representative steps presented as FIG. 2A. In some embodiments, for example, flowchart 200 may represent several exemplary steps that can be carried out by computer system 18 (e.g., by closed-loop ablation processor 20, with FIG. 2B illustrating exemplary inputs to and outputs from closed-loop ablation processor 20 in accordance with the following description). It should be understood that the representative steps illustrated in flowchart 200 and described below can be hardware-implemented (e.g., as an application-specific integrated circuit (ASIC), field programmable gate array (FPGA), or solid state electronics), software-implemented (e.g., as a series of programing instructions executed on one or more processing units), or in a combination of hardware and software.

In block 202, closed-loop ablation processor 20 receives baseline properties of a tissue to be ablated. The baseline tissue properties can include one or more of a baseline tissue impedance; the anatomical location of the tissue; the tissue type; the tissue thickness; the contact force between ablation catheter 12 and the tissue to be ablated (“contact force”); the stability of ablation catheter 12 against the tissue to be ablated (“catheter stability”); and the morphology of unipolar, bipolar, and/or omnipolar electrogram (EGM) signals at the tissue to be ablated.

Certain baseline tissue properties, such as baseline tissue impedance, the anatomical location of the tissue, contact force, catheter stability, and EGM signal morphology, may be measured using ablation catheter 12 according to techniques that will be familiar to those of ordinary skill in the art and that need not be described in detail herein. Other baseline tissue properties, such as tissue type and tissue thickness, may be externally provided, such as from a computed tomography (CT) image, an intracardiac echocardioagraphy (ICE) image, or the like.

In block 204, closed-loop ablation processor 20 receives ablation waveform parameters for ablation generator 16. For instance, with respect to PFA, ablation waveform parameters can include, without limitation, peak voltage amplitude, anodic and cathodic pulse widths, interphasic delay, intrapulse delay, number of pulses per burst, and intraburst delay. Similarly, with respect to RFA, ablation waveform parameters can include, without limitation, power, voltage, and/or current.

In block 206, a determination is made whether PFA or RFA therapy should be delivered to the tissue. This determination may be made automatically by closed-loop ablation processor 20 using the baseline tissue properties received in block 202, such as by using logic that takes into account tissue thickness, tissue anatomical location, and/or tissue type. For example, if the tissue anatomical location is the posterior left atrium, closed-loop ablation processor 20 can determine that PFA therapy should be delivered to the tissue. As another example, if the tissue anatomical location is an atrial location adjacent to a coronary artery, closed-loop ablation processor 20 can determine that RFA therapy should be delivered to the tissue. Of course, it is also within the scope of the instant disclosure for a practitioner to manually select between PFA and RFA therapies based on the practitioner's own knowledge and experience.

In block 208, closed-loop ablation processor 20 computes one or more target ablation metrics using the baseline tissue properties (received in block 202) and the ablation waveform parameters (received in block 204). Target ablation metrics represent the therapeutic endpoint—that is, the point at which it is likely that a suitable transmural ablation lesion has been created in the tissue.

Various target ablation metrics, which may be used either singly or in any combination, are contemplated. They are described in turn below.

### Target Ablation Index

In general, an ablation index (denoted herein PI for PFA therapy and RI for RFA therapy) is a multivariate function, the value of which is representative of lesion formation in a tissue. A target ablation index (denoted herein PI-T for PFA therapy and RI-T for RFA therapy) is the value of the ablation index at the therapeutic endpoint.

Closed-loop ablation processor 20 may compute PI-T or RI-T using a multivariate regression model, such as a linear, non-linear, or logistic regression model, where the input parameters are one or more of the baseline tissue properties and one or more of the ablation waveform parameters. The multivariate regression model may utilize logarithmic, trigonometric, exponential, and/or Gaussian functions to fit the input parameters. Nonlinear least square optimization methods (e.g., Levenberg-Marquardt and/or Gauss-Newton methods) are also contemplated to optimize model coefficients.

By way of example only, U.S. Pat. Nos. 9,149,327 and 11,883,106 , both of which are hereby incorporated by reference as though fully set forth herein, describe various models that may be suitable for computation of RI-T. Those of ordinary skill in the art will appreciate that similar techniques can be used to develop other models for the computation of RI-T, as well as models for the computation of PI-T.

### Target Impedance Drop

Another contemplated ablation metric, which may be used for both PFA and RFA therapy, is impedance drop (denoted ID). A target impedance drop (denoted ID-T) represents a prediction of the amount by which the impedance of the tissue being ablated will drop, relative to the baseline tissue impedance, upon reaching the therapeutic endpoint. The value of ID-T may be user-selected or computed by closed-loop ablation processor 20 (e.g., using the baseline tissue properties received in block 202).

### Target RFA Therapy Duration

Yet another contemplated ablation metric, which may be used for RFA therapy, is RFA therapy duration (denoted RD). A target RFA therapy duration (denoted RD-T) represents a prediction of the length of time over which RFA ablation therapy must be delivered to reach the therapeutic endpoint, given the ablation parameters. The value of RD-T may be user-selected or computed by closed-loop ablation processor 20 (e.g., using the ablation waveform parameters received in block 204 and baseline tissue properties received in block 202).

### Target EGM Signal Morphology Variations

Still other contemplated ablation metrics, which may be desirable in connection with the delivery of PFA therapy, are variations in the morphological characteristics (e.g., peak-to-peak amplitude, instantaneous energy, and so forth) of unipolar, bipolar, and/or omnipolar EGM signals. As those of ordinary skill in the art will appreciate, EGM signal morphology changes with formation of ablation lesions. By monitoring variations in EGM morphological characteristics (e.g., via analysis of EGM signal morphology over time), it is possible to predict when a transmural ablation lesion has been created.

Various EGM morphological characteristics may be used to define target EGM signal morphology variations. As one example, an attenuation in intra-therapeutic peak-to-peak EGM amplitude that is about 90% of the baseline (that is, pre-ablation) peak-to-peak EGM amplitude (in other words, the intra-therapeutic peak-to-peak EGM amplitude is about 90% lower than the baseline peak-to-peak EGM amplitude) may be used to define a target EGM signal morphology variation and therapy endpoint. As another example, an attenuation in intra-therapeutic instantaneous EGM signal energy that is about 90% of the baseline instantaneous EGM signal energy (in other words, the intra-therapeutic instantaneous EGM signal energy is about 90% lower than the baseline EGM signal energy) may be used to define a target EGM signal morphology variation and therapy endpoint. It should be understood, however, that these exemplary attenuation values are merely illustrative and that other attenuation values are contemplated.

It is also contemplated that the EGM signals may be decomposed and/or filtered (e.g., a low-frequency bandpass filter, such as between about 1 Hz and about 50 Hz, or between about 5 Hz and about 15 Hz, or between about 150 Hz and about 250 Hz may be applied to the EGM signals) for purposes of monitoring and analyzing morphology variations. It should be understood that the foregoing cutoff frequencies are merely exemplary and that those of ordinary skill in the art will appreciate that other cutoff frequencies may be desirable in particular contexts.

The foregoing target ablation metrics may be used singly or in any combination. For instance, in connection with delivery of RFA therapy, both RI-T and ID-T may be selected (manually by a practitioner or automatically by closed-loop ablation processor 20) as target ablation metrics. As another example, in connection with delivery of PFA therapy, both PI-T and ID-T may be selected (manually by a practitioner or automatically by closed-loop ablation processor 20) as target ablation metrics.

Once the target ablation metrics to be used for monitoring are selected and computed in block 208, closed-loop ablation processor 20 can optionally command ablation generator 16 to deliver ablation therapy (PFA or RFA, as determined in block 206) to the tissue (block 212). Commanding the ablation generator 16 to deliver ablation therapy may include the closed-loop ablation processor 20 sending a signal or instruction to ablation generator 16 to deliver ablation energy. It is contemplated that the practitioner may have “master arm” control over this command. That is, closed-loop ablation processor 20 can command ablation generator 16 to deliver ablation therapy only once affirmatively enabled to do so by the practitioner, such as by depressing a foot pedal or toggling another suitable enabling switch on (and vice-versa), once the practitioner has navigated catheter 12 to the therapy location.

During ablation, closed-loop ablation processor 20 monitors lesion formation. In general, the lesion formation monitoring process of block 214 includes receiving intra-therapeutic tissue properties (e.g., intra-therapeutic values for some or all of the same tissue properties for which baseline values were received in block 202), computing one or more intra-therapeutic ablation metrics from the received intra-therapeutic tissue properties, and comparing the intra-therapeutic ablation metric(s) to the target ablation metric(s) (e.g., PI-T/RI-T, ID-T, RD-T, and so forth) in order to titrate the delivery of ablation therapy (PFA or RFA) to the tissue. Since ablation processor 20 and computer system 18 are both configured to process data, the metric(s) may be digitized into data representative of the metric(s), as necessary. The closed-loop ablation processor 20 may determine whether ablation therapy is to be ceased when an intra-therapeutic ablation metric satisfies the target ablation metric(s). In some examples, this may further include commanding the ablation generator 16 to cease delivery of ablation therapy—for example, the closed-loop ablation processor 20 may send a signal or instruction to the ablation generator 16 to cease delivery of ablation energy. It is also contemplated that the practitioner may have “master disarm” control over the delivery of ablation energy. That is, the practitioner can cause the cessation of the delivery of ablation energy at any time, such as by releasing a foot pedal or toggling another suitable enabling switch off.

Nevertheless, there are certain variations in the lesion formation monitoring process of block 214 that depend upon whether ablation generator 16 is delivering PFA therapy or RFA therapy. The following examples serve to illustrate some of these variations to aid the skilled artisan in understanding the instant disclosure.

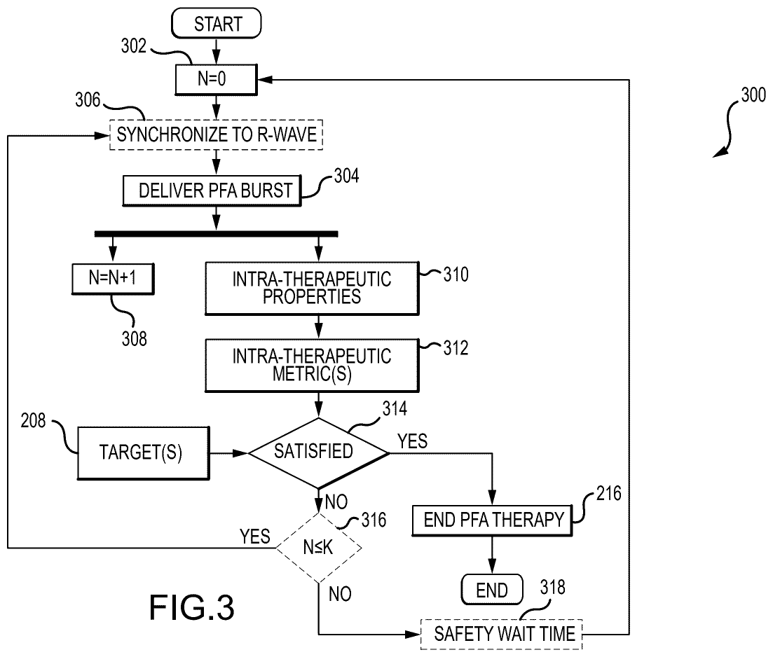

FIG. 3 is a flowchart 300 of exemplary steps that can be followed as part of the lesion formation monitoring process of block 214 when ablation generator 16 is delivering PFA therapy.

In block 302, a cycle counter (denoted N) is set to zero.

In block 304, a PFA burst (that is, a train of pulses) is delivered to the tissue. As shown in optional block 306, the PFA burst can be R-wave synchronized (e.g., delivered after an R-wave is detected by the ECG monitor within electronics 14), if desired.

According to the embodiment shown in FIG. 3, lesion formation monitoring occurs between PFA bursts (that is, during the intraburst interval). In block 308, the cycle counter Nis incremented by one to reflect the PFA burst delivered in block 304.

In parallel, as shown in block 310, closed-loop ablation processor 20 receives intra-therapeutic tissue properties (e.g., one or more of intra-therapeutic tissue impedance, intra-therapeutic contact force, intra-therapeutic catheter stability, and intra-therapeutic EGM signal morphology; the intra-therapeutic tissue properties may optionally further include tissue anatomical location, tissue thickness, and/or tissue type). These intra-therapeutic tissue properties (as well as the ablation waveform parameters) are used, in block 312, to compute one or more intra-therapeutic ablation metrics corresponding to the target metric(s) selected and computed for lesion formation monitoring purposes in block 208.

To illustrate, an intra-therapeutic ablation index (denoted PI-B) may be computed in block 312 from the intra-therapeutic tissue properties received in block 310 using the same multivariate regression model used to compute PI-T from the baseline tissue properties and ablation waveform parameters in block 208. Similarly, an intra-therapeutic impedance drop (that is, the amount by which the intra-therapeutic tissue impedance received in block 310 has dropped relative to the baseline tissue impedance in block 202) can be computed in block 312.

In decision block 314, the intra-therapeutic ablation metric(s) computed in block 312 may be compared to corresponding target ablation metric(s) from block 208 to determine whether all selected target ablation metric(s) are satisfied. If all target ablation metric(s) are satisfied (e.g., the “YES” exit from decision block 314), closed-loop ablation processor 20 can command ablation generator 16 to cease delivering ablation therapy to the tissue in block 216.

For instance, PI-B (computed in block 312) may be compared to PI-T (computed in block 208) in decision block 314. If PI-B meets or exceeds PI-T, then the target ablation metric PI-T is satisfied.

As another example, the intra-therapeutic impedance drop may be compared to ID-T (computed in block 208). If the intra-therapeutic impedance drop meets or exceeds ID-T, then the target ablation metric ID-T is satisfied.

Where target ablation metrics are applied in combination decision block 314 will evaluate each intra-therapeutic metric relative to its corresponding target ablation metric, and will proceed to the “YES” exit only if all evaluated intra-therapeutic ablation metrics satisfy the corresponding target ablation metrics.

On the other hand, if all intra-therapeutic ablation metric(s) do not satisfy corresponding target ablation metric(s) (the “NO” exit from decision block 314), then flowchart 300 can proceed to optional decision block 316 to determine whether the cycle counter N, which reflects the number of PFA therapy bursts that have been delivered to the tissue, exceeds a preset threshold k. The specific value of k may be dependent on various factors, such as the ablation waveform parameters, the specific PFA catheter being used in a given procedure, and so forth, and may also be selected or adjusted by the practitioner. By way of example only, for the TactiFlex™ Duo Ablation Catheter, Sensor Enabled™ mentioned above, k may be preset to a value between 5 and 10 cycles.

If N does not exceed k (the “YES” exit from decision block 316), then an additional burst of (optionally R-wave synchronized in block 306) PFA therapy can be delivered through a return to block 304.

If N does exceed k (the “NO” exit from decision block 316), then delivery of PFA therapy bursts can be suspended for a safety wait time (block 318) before returning to block 302 and resetting the cycle counter N prior to delivery of additional PFA therapy bursts. Like k, the specific length of the safety wait time may be dependent on various factors, such as the ablation waveform parameters, the specific PFA catheter being used in a given procedure, and so forth, and may also be selected or adjusted by the practitioner. By way of example only, for the TactiFlex™ Duo Ablation Catheter, Sensor Enabled™ mentioned above, the safety wait time may be preset to a value between about 1 second and about 10 seconds.

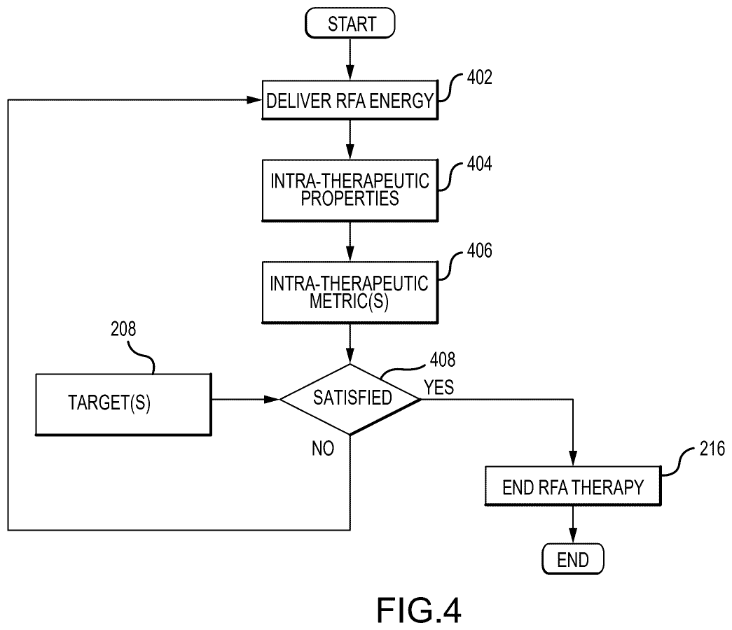

FIG. 4 is a flowchart 400 of exemplary steps that can be followed as part of the lesion formation monitoring process of block 214 when ablation generator 16 is delivering RFA therapy.

In block 402, ablation generator 16 begins to deliver RFA therapy to the tissue. As those of ordinary skill in the art will appreciate, because RFA therapy is delivered continuously, rather than in bursts as in the case of PFA therapy, lesion formation monitoring in FIG. 4 occurs simultaneous with the delivery of RFA therapy. Thus, in block 404, closed-loop ablation processor 20 receives intra-therapeutic tissue properties (e.g., one or more of intra-therapeutic tissue impedance, intra-therapeutic contact force, intra-therapeutic catheter stability, and intra-therapeutic EGM signal morphology; the intra-therapeutic tissue properties may optionally further include tissue anatomical location, tissue thickness, and/or tissue type). These intra-therapeutic tissue properties (as well as the ablation waveform parameters) are used, in block 406, to compute one or more intra-therapeutic ablation metrics corresponding to the target metric(s) selected and computed for lesion formation monitoring purposes in block 208.

To illustrate, an intra-therapeutic ablation index (denoted RI) may be computed in block 406 from the intra-therapeutic tissue properties received in block 404 using the same multivariate regression model used to compute RI-T from the baseline tissue properties and ablation waveform parameters in block 208. Similarly, an intra-therapeutic impedance drop (that is, the amount by which the intra-therapeutic tissue impedance received in block 404 has dropped relative to the baseline tissue impedance in block 202) can be computed in block 406.

In decision block 408, the intra-therapeutic ablation metric(s) computed in block 406 may be compared to corresponding target ablation metric(s) from block 208 to determine whether all selected target ablation metric(s) are satisfied. If all target ablation metric(s) are satisfied (e.g., the “YES” exist from decision block 408), closed-loop ablation processor 20 can command ablation generator 16 to cease delivering ablation therapy to the tissue in block 216.

For instance, RI (computed in block 406) may be compared to RI-T (computed in block 208) in decision block 408. If RI meets or exceeds RI-T, then the target ablation metric RI-T is satisfied.

As another example, the intra-therapeutic impedance drop may be compared to ID-T (computed in block 208). If the intra-therapeutic impedance drop meets or exceeds ID-T, then the target ablation metric ID-T is satisfied.

Where target ablation metrics are applied in combination, decision block 408 will evaluate each intra-therapeutic metric relative to its corresponding target ablation metric, and will proceed to the “YES” exit only if all evaluated intra-therapeutic ablation metrics satisfy the corresponding target ablation metrics.

On the other hand, if all intra-therapeutic ablation metric(s) do not satisfy corresponding target ablation metric(s) (the “NO” exit from decision block 408), then flowchart 400 loops back to block 402 for continuing delivery of RFA therapy to the tissue. (Note that, although the loop back to block 402 might suggest that RFA therapy is delivered in increments, that is not the case; as mentioned above, RFA therapy is delivered continuously with lesion formation monitoring occurring in parallel.)

In accordance with the foregoing teachings, the delivery of ablation therapy can be titrated, customized, and individualized to particular patients while maintaining a high degree of confidence (about 95% or higher) for the creation of transmural lesions.

Although several embodiments have been described above with a certain degree of particularity, those skilled in the art could make numerous alterations to the disclosed embodiments without departing from the spirit or scope of this invention.

For instance, it is contemplated that closed-loop ablation processor 20 may be part of ablation generator 16, rather than part of a separate computer system 18 operably coupled thereto.

For completeness, the methods described herein may be methods that are embedded within a set of instructions that are comprised within a computer-readable medium or record carrier, or that are comprised within a computer program product. The instructions are such that, when executed by a computer or processor, such as the ablation processor as described herein, or the CPU of the computer system described herein, or a processor of a general-purpose computer system, the computer or processor causes the system to perform the methods described herein.

All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other.

It is intended that all matter contained in the above description or shown in the accompanying drawings shall be interpreted as illustrative only and not limiting. Changes in detail or structure may be made without departing from the spirit of the invention as defined in the appended claims.

Aspects of the disclosure are as set out in the following numbered clauses:
1. A computer-implemented method of determining, by a closed-loop ablation processor, ablation therapy to be delivered to a tissue via an ablation system, the ablation system including an ablation generator operably coupled to the closed-loop ablation processor, the method comprising:
   receiving baseline properties of the tissue as inputs, the baseline properties of the tissue including one or more of: a baseline tissue impedance, a tissue thickness, a tissue anatomical location, a tissue type, a contact force between the ablation catheter and the tissue, a stability of the ablation catheter against the tissue, and a baseline electrogram (EGM) signal morphology;
   receiving ablation waveform parameters for the ablation generator as inputs;
   computing a target ablation metric using the baseline properties of the tissue and the ablation waveform parameters;
   executing a lesion formation monitoring process comprising:
      receiving intra-therapeutic properties of the tissue as input, the intra-therapeutic properties of the tissue including one or more of an intra-therapeutic tissue impedance, an intra-therapeutic contact force, an intra-therapeutic catheter stability, and an intra-therapeutic EGM signal morphology;
      computing an intra-therapeutic ablation metric using the intra-therapeutic properties of the tissue; and
      comparing the intra-therapeutic ablation metric to the target ablation metric; and
      determining that ablation therapy to the tissue should be ceased when the intra-therapeutic ablation metric satisfies the target ablation metric.
2. The method according to clause 1, further comprising commanding the ablation generator to cease delivering ablation therapy to the tissue when the intra-therapeutic ablation metric satisfies the target ablation metric.
3. The method according to clause 1 or 2, further comprising after computing the target ablation metric, the closed-loop ablation processor commanding the ablation generator to deliver ablation therapy to the tissue;
4. The method according to clause 1, 2 or 3, wherein the target ablation metric comprises one or more of a target ablation index, a target impedance drop, a target radiofrequency ablation (RFA) duration, and a target EGM signal morphology variation.
5. The method according to clause 4, wherein the target ablation index is computed according to a multivariate regression model wherein variables of the multivariate regression model correspond to one or more of the baseline properties of the tissue and the ablation waveform parameters.
6. The method according to clause 5, wherein the multivariate regression model comprises a multivariate logistic model.
7. The method according to any of the previous clauses, further comprising, the closed-loop ablation processor determining, based on the baseline properties of the tissue, either to command the ablation generator to deliver RFA therapy to the tissue or to command the ablation generator to deliver pulsed field ablation therapy to the tissue.
8. The method according to clause 7, wherein the closed-loop ablation processor determining, based on the baseline properties of the tissue, either to command the ablation generator to deliver RFA therapy to the tissue or to command the ablation generator to deliver pulsed field ablation therapy to the tissue comprises the closed-loop ablation processor determining, based on one or more of the tissue thickness, the tissue anatomical location, and the tissue type, either to command the ablation generator to deliver RFA therapy to the tissue or to command the ablation generator to deliver pulsed field ablation therapy to the tissue.
9. The method according to any of the previous clauses, wherein the intra-therapeutic properties of the tissue further comprise one or more of the tissue thickness, the tissue anatomical location, and the tissue type.
10. The method according to any of the previous clauses, wherein the ablation therapy comprises pulsed field ablation therapy, and wherein the closed-loop ablation processor executes the lesion formation monitoring process during intervals between successive bursts of pulsed field ablation therapy.
11. The method according to clause 10, wherein the pulsed field ablation therapy is R-wave synchronized.
12. The method according to clause 10 or 11, further comprising the closed-loop ablation processor determining that the ablation generator is to suspend delivery of ablation therapy to the tissue for a preset safety interval after a preset number of successive bursts of pulsed field ablation therapy are delivered to the tissue.
13. A closed-loop ablation system, comprising:
   an ablation generator;
   an ablation catheter operably coupled to the ablation generator; and
   a closed-loop ablation processor operably coupled to the ablation generator, wherein the closed-loop ablation processor is configured to:
      receive baseline properties of a tissue to be ablated as inputs, the baseline properties of the tissue including one or more of: a baseline tissue impedance, a tissue thickness, a tissue anatomical location, a tissue type, a contact force between the ablation catheter and the tissue, a stability of the ablation catheter against the tissue, and a baseline electrogram (EGM) signal morphology;
      receive ablation waveform parameters for the ablation generator as inputs;
      compute a target ablation metric using the baseline properties of the tissue and the ablation waveform parameters;
      command the ablation generator to deliver ablation therapy to the tissue after computing the target ablation metric;
      execute a lesion formation monitoring process after commanding the ablation generator to deliver ablation therapy to the tissue, the lesion formation monitoring processing comprising:
         receiving intra-therapeutic properties of the tissue as inputs, the intra-therapeutic properties of the tissue including one or more of an intra-therapeutic tissue impedance, an intra-therapeutic contact force, an intra-therapeutic catheter stability, and an intra-therapeutic EGM signal morphology;
         computing an intra-therapeutic ablation metric using the intra-therapeutic properties of the tissue; and
         comparing the intra-therapeutic ablation metric to the target ablation index; and
      command the ablation generator to cease delivering ablation therapy to the tissue when the intra-therapeutic ablation metric satisfies the target ablation metric.
14. The closed-loop ablation system according to clause 13, wherein the target ablation metric comprises one or more of a target ablation index, a target impedance drop, a target radiofrequency ablation (RFA) duration, and a target EGM signal morphology variation.
15. The closed-loop ablation system according to clause 14, wherein the target ablation index is computed according to a multivariate regression model wherein variables of the multivariate regression model correspond to one or more of the baseline properties of the tissue and the ablation waveform parameters.
16. The closed-loop ablation system according to clause 15, wherein the multivariate regression model comprises a multivariate logistic model
17. The closed-loop ablation system according to any of clauses 13 to 16, wherein the closed-loop ablation processor is further configured to determine, based on the baseline properties of the tissue, either to command the ablation generator to deliver RFA therapy to the tissue or to command the ablation generator to deliver pulsed field ablation therapy to the tissue.
18. The closed-loop ablation system according to clause 17, wherein the closed-loop ablation processor is further configured to determine, based on at least one of the tissue thickness, the tissue anatomical location, and the tissue type, either to command the ablation generator to deliver RFA therapy to the tissue or to command the ablation generator to deliver pulsed field ablation therapy to the tissue.
19. The closed-loop ablation system according to any of clauses 13 to 18, wherein the ablation therapy comprises pulsed field ablation therapy and wherein the closed-loop ablation processor executes the lesion formation monitoring process during intervals between successive bursts of pulsed field ablation therapy.
20. The closed-loop ablation system according to any of clauses 13 to 19, wherein the ablation therapy comprises pulsed field ablation therapy and wherein the pulsed-field ablation therapy is R-wave synchronized.
21. The closed-loop ablation system according to any of clauses 13 to 20, wherein the ablation therapy comprises pulsed field ablation therapy and wherein the closed-loop ablation processor is further configured to command the ablation generator to suspend delivery of the ablation therapy to the tissue for a preset safety interval after a preset number of successive bursts of pulsed field ablation therapy are delivered to the tissue.
22. The closed-loop ablation system according to any of clauses 13 to 21, wherein the intra-therapeutic properties of the tissue further comprise one or more of the tissue thickness, the tissue anatomical location, and the tissue type.
23. A method of determining, by a closed-loop ablation processor ablation processor, ablation therapy to be delivered to a tissue via an ablation system, the ablation system including an ablation generator operably coupled to the closed-loop ablation processor, the method comprising:
   receiving baseline properties of the tissue, the baseline properties of the tissue including one or more of: a baseline tissue impedance, a tissue thickness, a tissue anatomical location, a tissue type, a contact force between the ablation catheter and the tissue, a stability of the ablation catheter against the tissue, and a baseline electrogram (EGM) signal morphology;
   receiving ablation waveform parameters for the ablation generator;
   selecting between radiofrequency ablation therapy and pulsed field ablation therapy based on the baseline properties of the tissue;
   computing a target ablation index using a multivariate regression model wherein variables of the multivariate regression model correspond to one or more of the baseline properties of the tissue and one or more of the ablation waveform parameters; and
   monitoring a relationship between an intra-therapeutic ablation index and the target ablation index, wherein the intra-therapeutic ablation index is a function of one or more intra-therapeutic properties of the tissue for titrating delivery of the selected ablation therapy.
24. The method according to clause 23, further comprising the closed-loop ablation processor monitoring a drop in tissue impedance relative to the baseline tissue impedance for titrating delivery of the selected ablation therapy.
25. The method according to clause 23 or 24 further comprising commanding the ablation generator to deliver the determined ablation therapy to the tissue.
26 A method of determining, by a closed-loop ablation processor, ablation therapy to be delivered to a tissue via an ablation system, the ablation system including an ablation generator operably coupled to the closed-loop ablation processor, the method comprising
   receiving baseline properties of the tissue as inputs, the baseline properties of the tissue including one or more of: a baseline tissue impedance, a tissue thickness, a tissue anatomical location, a tissue type, a contact force between the ablation catheter and the tissue, a stability of the ablation catheter against the tissue, and a baseline electrogram (EGM) signal morphology; and
   determining, based on the baseline properties of the tissue, either to command the ablation generator to deliver radiofrequency ablation therapy to the tissue or to command the ablation generator to deliver pulsed field ablation therapy to the tissue, and then commanding the ablation generator to deliver the determined ablation therapy to the tissue.
27. A computer program which, when the program is executed by a computer, cause the computer to carry out the method of any of clauses 1 to 12 and 23 to 26.

What is claimed is:

1. A method of delivering closed-loop ablation therapy to a tissue via an ablation system including an ablation generator and a closed-loop ablation processor operably coupled to the ablation generator, the method comprising:
receiving baseline properties of the tissue as inputs to the closed-loop ablation processor, the baseline properties of the tissue including one or more of: a baseline tissue impedance, a tissue thickness, a tissue anatomical location, a tissue type, a contact force between the ablation catheter and the tissue, a stability of the ablation catheter against the tissue, and a baseline electrogram (EGM) signal morphology;
receiving ablation waveform parameters for the ablation generator as inputs to the closed-loop ablation processor;
the closed-loop ablation processor computing a target ablation metric using the baseline properties of the tissue and the ablation waveform parameters;
after computing the target ablation metric, the closed-loop ablation processor commanding the ablation generator to deliver ablation therapy to the tissue;
after commanding the ablation generator to deliver ablation therapy to the tissue, the closed-loop ablation processor executing a lesion formation monitoring process comprising:
receiving intra-therapeutic properties of the tissue as input, the intra-therapeutic properties of the tissue including one or more of an intra-therapeutic tissue impedance, an intra-therapeutic contact force, an intra-therapeutic catheter stability, and an intra-therapeutic EGM signal morphology;
computing an intra-therapeutic ablation metric using the intra-therapeutic properties of the tissue; and
comparing the intra-therapeutic ablation metric to the target ablation metric; and
the closed-loop ablation processor commanding the ablation generator to cease delivering ablation therapy to the tissue when the intra-therapeutic ablation metric satisfies the target ablation metric.

2. The method according to claim 1, wherein the target ablation metric comprises one or more of a target ablation index, a target impedance drop, a target radiofrequency ablation (RFA) duration, and a target EGM signal morphology variation.

3. The method according to claim 2, wherein the target ablation index is computed according to a multivariate regression model wherein variables of the multivariate regression model correspond to one or more of the baseline properties of the tissue and the ablation waveform parameters.

4. The method according to claim 3, wherein the multivariate regression model comprises a multivariate logistic model.

5. The method according to claim 1, further comprising, prior to the closed-loop ablation processor commanding the ablation generator to deliver ablation therapy to the tissue, the closed-loop ablation processor determining, based on the baseline properties of the tissue, either to command the ablation generator to deliver RFA therapy to the tissue or to command the ablation generator to deliver pulsed field ablation therapy to the tissue.

6. The method according to claim 5, wherein the closed-loop ablation processor determining, based on the baseline properties of the tissue, either to command the ablation generator to deliver RFA therapy to the tissue or to command the ablation generator to deliver pulsed field ablation therapy to the tissue comprises the closed-loop ablation processor determining, based on one or more of the tissue thickness, the tissue anatomical location, and the tissue type, either to command the ablation generator to deliver RFA therapy to the tissue or to command the ablation generator to deliver pulsed field ablation therapy to the tissue.

7. The method according to claim 1, wherein the intra-therapeutic properties of the tissue further comprise one or more of the tissue thickness, the tissue anatomical location, and the tissue type.

8. The method according to claim 1, wherein the ablation therapy comprises pulsed field ablation therapy, and wherein the closed-loop ablation processor executes the lesion formation monitoring process during intervals between successive bursts of pulsed field ablation therapy.

9. The method according to claim 8, wherein the pulsed field ablation therapy is R-wave synchronized.

10. The method according to claim 8, further comprising the closed-loop ablation processor commanding the ablation generator to suspend delivery of ablation therapy to the tissue for a preset safety interval after a preset number of successive bursts of pulsed field ablation therapy are delivered to the tissue.

11. A closed-loop ablation system, comprising:
an ablation generator;
an ablation catheter operably coupled to the ablation generator; and
a closed-loop ablation processor operably coupled to the ablation generator, wherein the closed-loop ablation processor is configured to:
receive baseline properties of a tissue to be ablated as inputs, the baseline properties of the tissue including one or more of: a baseline tissue impedance, a tissue thickness, a tissue anatomical location, a tissue type, a contact force between the ablation catheter and the tissue, a stability of the ablation catheter against the tissue, and a baseline electrogram (EGM) signal morphology;
receive ablation waveform parameters for the ablation generator as inputs;
compute a target ablation metric using the baseline properties of the tissue and the ablation waveform parameters;
command the ablation generator to deliver ablation therapy to the tissue after computing the target ablation metric;
execute a lesion formation monitoring process after commanding the ablation generator to deliver ablation therapy to the tissue, the lesion formation monitoring processing comprising:
receiving intra-therapeutic properties of the tissue as inputs, the intra-therapeutic properties of the tissue including one or more of an intra-therapeutic tissue impedance, an intra-therapeutic contact force, an intra-therapeutic catheter stability, and an intra-therapeutic EGM signal morphology;
computing an intra-therapeutic ablation metric using the intra-therapeutic properties of the tissue; and
comparing the intra-therapeutic ablation metric to the target ablation index; and
command the ablation generator to cease delivering ablation therapy to the tissue when the intra-therapeutic ablation metric satisfies the target ablation metric.

12. The closed-loop ablation system according to claim 11, wherein the target ablation metric comprises one or more of a target ablation index, a target impedance drop, a target radiofrequency ablation (RFA) duration, and a target EGM signal morphology variation.

13. The closed-loop ablation system according to claim 12, wherein the target ablation index is computed according to a multivariate regression model wherein variables of the multivariate regression model correspond to one or more of the baseline properties of the tissue and the ablation waveform parameters.

14. The closed-loop ablation system according to claim 13, wherein the multivariate regression model comprises a multivariate logistic model.

15. The closed-loop ablation system according to claim 11, wherein the closed-loop ablation processor is further configured to determine, based on the baseline properties of the tissue, either to command the ablation generator to deliver RFA therapy to the tissue or to command the ablation generator to deliver pulsed field ablation therapy to the tissue.

16. The closed-loop ablation system according to claim 15, wherein the closed-loop ablation processor is further configured to determine, based on at least one of the tissue thickness, the tissue anatomical location, and the tissue type, either to command the ablation generator to deliver RFA therapy to the tissue or to command the ablation generator to deliver pulsed field ablation therapy to the tissue.

17. The closed-loop ablation system according to claim 11, wherein the ablation therapy comprises pulsed field ablation therapy and wherein the closed-loop ablation processor executes the lesion formation monitoring process during intervals between successive bursts of pulsed field ablation therapy.

18. The closed-loop ablation system according to claim 11, wherein the ablation therapy comprises pulsed field ablation therapy and wherein the pulsed-field ablation therapy is R-wave synchronized.

19. The closed-loop ablation system according to claim 11, wherein the ablation therapy comprises pulsed field ablation therapy and wherein the closed-loop ablation processor is further configured to command the ablation generator to suspend delivery of the ablation therapy to the tissue for a preset safety interval after a preset number of successive bursts of pulsed field ablation therapy are delivered to the tissue.

20. The closed-loop ablation system according to claim 11, wherein the intra-therapeutic properties of the tissue further comprise one or more of the tissue thickness, the tissue anatomical location, and the tissue type.

21. A method of delivering closed-loop ablation therapy to a tissue via a dual-mode ablation system including an ablation generator, an ablation catheter operably coupled to the ablation generator, and a closed-loop ablation processor operably coupled to the ablation generator, the method comprising:
the closed-loop ablation processor receiving baseline properties of the tissue, the baseline properties of the tissue including one or more of: a baseline tissue impedance, a tissue thickness, a tissue anatomical location, a tissue type, a contact force between the ablation catheter and the tissue, a stability of the ablation catheter against the tissue, and a baseline electrogram (EGM) signal morphology;
the closed-loop ablation processor receiving ablation waveform parameters for the ablation generator;
the closed-loop ablation processor selecting between radiofrequency ablation therapy and pulsed field ablation therapy based on the baseline properties of the tissue;
the closed-loop ablation processor computing a target ablation index using a multivariate regression model wherein variables of the multivariate regression model correspond to one or more of the baseline properties of the tissue and one or more of the ablation waveform parameters; and
the closed-loop ablation processor titrating delivery of the selected ablation therapy by monitoring a relationship between an intra-therapeutic ablation index and the target ablation index, wherein the intra-therapeutic ablation index is a function of one or more intra-therapeutic properties of the tissue.

22. The method according to claim 21, further comprising the closed-loop ablation processor titrating delivery of the selected ablation therapy by monitoring a drop in tissue impedance relative to the baseline tissue impedance.

23. A method of delivering closed-loop ablation therapy to a tissue via an ablation system including an ablation generator and a closed-loop ablation processor operably coupled to the ablation generator, the method comprising:
receiving baseline properties of the tissue as inputs to the closed-loop ablation processor, the baseline properties of the tissue including one or more of: a baseline tissue impedance, a tissue thickness, a tissue anatomical location, a tissue type, a contact force between the ablation catheter and the tissue, a stability of the ablation catheter against the tissue, and a baseline electrogram (EGM) signal morphology; and
the closed-loop ablation processor determining, based on the baseline properties of the tissue, either to command the ablation generator to deliver radiofrequency ablation therapy to the tissue or to command the ablation generator to deliver pulsed field ablation therapy to the tissue, and then commanding the ablation generator to deliver the determined ablation therapy to the tissue.
